(19) United States
(12) Patent Application Publication
(10) Pub. No.: US 2025/0274666 A1
(43) Pub. Date: Aug. 28, 2025

(54) MEDICAL CONTROL DEVICE AND MEDICAL OBSERVATION SYSTEM

(71) Applicant: Sony Olympus Medical Solutions Inc., Tokyo (JP)

(72) Inventor: Koji KOJIMA, Tokyo (JP)

(73) Assignee: Sony Olympus Medical Solutions Inc., Tokyo (JP)

(21) Appl. No.: 19/052,490

(22) Filed: Feb. 13, 2025

(30) Foreign Application Priority Data
Feb. 22, 2024 (JP) 2024-025712

(51) Int. Cl. H04N 23/72; A61B 1/04; A61B 1/045; A61B 5/00; G03B 7/00; H04N 23/50; H04N 23/56; H04N 23/62; H04N 23/667; H04N 23/73; H04N 23/74; H04N 23/76

(52) U.S. Cl. CPC H04N 23/72; A61B 1/043; A61B 1/045; A61B 5/0071; G03B 7/00; H04N 23/56; H04N 23/62; H04N 23/667; H04N 23/73; H04N 23/74; H04N 23/76; H04N 23/555

(57) ABSTRACT

A medical control device includes: an imaging control unit configured to control an operation of an imaging element, and cause the imaging element to capture first return light from an observation target irradiated with first light that is narrow band light and second return light from the observation target irradiated with second light having a wavelength band different from a wavelength band of the narrow band light. The imaging control unit includes a shutter control unit configured to change a signal value related to a first image by controlling an opening amount of a shutter for imaging, the first image being generated by the imaging element when the first return light is captured. The shutter control unit is configured to change the opening amount of the shutter from a first opening amount to a second opening amount smaller than the first opening amount.

### CROSS-REFERENCE TO RELATED APPLICATION

This application claims priority from Japanese Application No. 2024-025712, filed on Feb. 22, 2024, the contents of which are incorporated by reference herein in its entirety.

### BACKGROUND

The present disclosure relates to a medical control device and a medical observation system.

In the related art, there has been known a medical observation system that irradiates an observation target (subject such as a person) with excitation light that is narrow band light emitted from a light source device and observes fluorescence emitted from a substance included in the observation target by irradiation with the excitation light (see, for example, JP 2023-119524 A).

According to such fluorescence observation, a tissue state that is difficult to recognize may be grasped via fluorescence. Therefore, fluorescence observation may be used for various purposes and applications such as identification of a lesion.

### SUMMARY

Intensity of fluorescence at the time of fluorescence observation varies depending on the difference in conditions regarding the fluorescence observation. For example, as an agent to be administered to an observation target, there are a first agent having weak intensity of fluorescence to be emitted and a second agent having high intensity of fluorescence to be emitted. When the first agent is used, the intensity of fluorescence may be increased (an image obtained through capturing of fluorescence may be brightened) by signal processing (increase of an analog gain or a digital gain). On the other hand, when the second agent is used, a signal value of the image is saturated when fluorescence is captured, and thus, even if signal processing (reduction of an analog gain or a digital gain) is simply performed, gradation of the image is lost.

Therefore, there is a demand for a technique capable of generating an image suitable for observation, and so there is a need for a medical control device and a medical observation system capable of generating an image suitable for observation.

According to one aspect of the present disclosure, there is provided a medical control device including: an imaging control unit configured to control an operation of an imaging element, and cause the imaging element to capture first return light from an observation target irradiated with first light that is narrow band light and second return light from the observation target irradiated with second light having a wavelength band different from a wavelength band of the narrow band light, wherein the imaging control unit includes a shutter control unit configured to change a signal value related to a first image by controlling an opening amount of a shutter for imaging, the first image being generated by the imaging element when the first return light is captured, and the shutter control unit is configured to change the opening amount of the shutter from a first opening amount to a second opening amount smaller than the first opening amount to set a ratio of the signal value related to the first image to a signal value related to a second image to a second ratio smaller than a first ratio, where the first ratio is a ratio of the signal value related to the first image to the signal value related to the second image, the second image being generated by the imaging element when the second return light is captured when the opening amount of the shutter is the first opening amount.

According to another aspect of the present disclosure, there is provided a medical control device including: an imaging control unit configured to control an operation of an imaging element and causes the imaging element to capture return light from an observation target irradiated with narrow band light, wherein the imaging control unit includes a shutter control unit configured to change a signal value related to an image by controlling an opening amount of a shutter for imaging, the image being generated by the imaging element when the return light is captured, and the shutter control unit is configured to set the opening amount of the shutter to a second opening amount smaller than a first opening amount to desaturate the signal value when the signal value is saturated in a case where the opening amount of the shutter is set to the first opening amount.

According to still another aspect of the present disclosure, there is provided a medical observation system including: an imaging element configured to capture first return light from an observation target irradiated with first light that is narrow band light and second return light from the observation target irradiated with second light having a wavelength band different from a wavelength band of the narrow band light; and an imaging control unit configured to control an operation of the imaging element, wherein the imaging control unit includes a shutter control unit configured to change a signal value related to a first image by controlling an opening amount of a shutter for imaging, the first image being generated by the imaging element when the first return light is captured, and the shutter control unit is configured to change the opening amount of the shutter from a first opening amount to a second opening amount smaller than the first opening amount to set a ratio of the signal value related to the first image to a signal value related to a second image to a second ratio smaller than a first ratio, where the first ratio is a ratio of the signal value related to the first image to the signal value related to the second image, the second image being generated by the imaging element when the second return light is captured when the opening amount of the shutter is the first opening amount.

According to yet another aspect of the present disclosure, there is provided a medical observation system including: an imaging element configured to capture return light from an observation target irradiated with narrow band light; and an imaging control unit configured to control an operation of the imaging element, wherein the imaging control unit includes a shutter control unit configured to change a signal value related to an image by controlling an opening amount of a shutter for imaging, the image being generated by the imaging element when the return light is captured, and the shutter control unit is configured to set the opening amount of the shutter to a second opening amount smaller than a first opening amount to desaturate the signal value when the signal value is saturated in a case where the opening amount of the shutter is set to the first opening amount.

### BRIEF DESCRIPTION OF THE DRAWINGS

FIG. 1 is a diagram illustrating a configuration of a medical observation system according to an embodiment;

FIG. 2 is a block diagram illustrating configurations of a camera head and a control device;

FIG. 3 is a diagram for explaining a problem;

FIG. 4 is a diagram for explaining gain control, light source control, and electronic shutter control according to the embodiment;

FIG. 5 is a table illustrating fluorescence gains set by a user;

FIG. 6 is a diagram for explaining a first modification of the embodiment;

FIG. 7 is a diagram for explaining the first modification of the embodiment;

FIG. 8 is a diagram for explaining the first modification of the embodiment;

FIG. 9 is a diagram for explaining the first modification of the embodiment;

FIG. 10 is a diagram for explaining the first modification of the embodiment;

FIG. 11 is a table for explaining the first modification of the embodiment;

FIG. 12 is a view for explaining a second modification of the embodiment;

FIG. 13 is a diagram for explaining a third modification of the embodiment;

FIG. 14 is a view for explaining a fourth modification of the embodiment; and

FIG. 15 is a view for explaining the fourth modification of the embodiment.

### DETAILED DESCRIPTION

Hereinafter, embodiments for carrying out the present disclosure (hereinafter referred to as embodiments) will be described with reference to the drawings. Note that the present disclosure is not limited by the embodiments described below. Moreover, in the description of the drawings, the same parts are denoted by the same reference numerals.

### Configuration of Medical Observation System

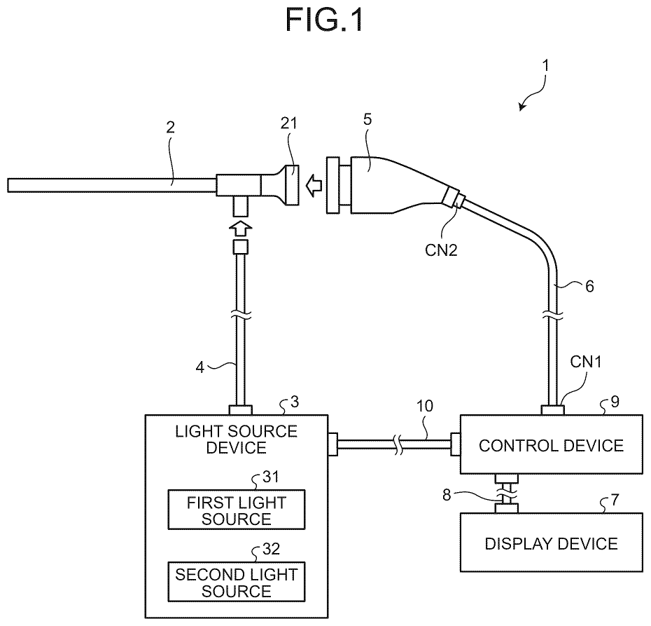

FIG. 1 is a diagram illustrating a configuration of a medical observation system 1 according to an embodiment. In the present embodiment, the medical observation system 1 is a medical endoscope system that observes an observation target (inside of a living body) using an endoscope. As illustrated in FIG. 1, the medical observation system 1 includes an insertion unit 2, a light source device 3, a light guide 4, a camera head 5, a first transmission cable 6, a display device 7, a second transmission cable 8, a control device 9, and a third transmission cable 10.

In the present embodiment, the insertion unit 2 is constituted by a rigid endoscope. That is, the insertion unit 2 has an elongated shape, which is entirely rigid or partially flexible and partially rigid, and is inserted into an observation target. An optical system that is composed of one or a plurality of lenses and condenses return light (subject image) from the observation target is provided in the insertion unit 2.

The light source device 3 is connected to a first end of the light guide 4 and includes a first light source 31 (FIG. 1) that supplies first light to the first end of the light guide 4 and a second light source 32 (FIG. 1) that supplies second light to the first end of the light guide 4 under the control of the control device 9. In the present embodiment, the first light is excitation light that is narrow band light. Note that the first light may be visible light or invisible light. In addition, the second light is white light including a visible wavelength band. In addition, the first light source 31 may be constituted by a light emitting diode (LED) or a semiconductor laser. Similarly, the second light source 32 may be constituted by an LED or a semiconductor laser. In addition, the number of the first light sources 31 that emit the first light may be one or more. Similarly, the number of the second light sources 32 that emit the second light may be one or more.

Here, examples of a substance included in the observation target excited by the first light include an agent or a fluorescent dye given to the observation target, or a fluorescent substance derived from the observation target forming the observation target itself.

Examples of the above-described agent to be given to the observation target include “5-ALA (PP-IX)”, “ADS780WS”, “ADS 830WS”, “aggregation-induced emission dots allophycocyanin (APC)”, “boron-dipyrromethane (BODIPY)”, “CLR 1502”, “Flavins”, “fluorescamine”, “fluorescein”, “fluoro-gold”, “green fluorescence protein”, “ICG (indocyanine green)”, “IRDye78”, “IR-PEG nanoparticles”, “Isothiocyanate”, “rose Bengal”, “SGM-101”, and “trypan blue”.

In addition, examples of the above-described fluorescent dye to be given to the observation target include “coumarine”, “Cy3”, “DyLight547”, “GE3126”, “metal nanoclusters”, “oxacarbocyanine”, “Rhodamine”, “Riboflavin”, “fluorescein”, “AlexaFluor488”, “AlexaFluor660”, “AlexaFluor680”, “AlexaFluor700”, “Cy5”, “Cy5.5”, “Dy677”, “Dy682”, “Dy752”, “DyLight647”, “HiLyte Fluor647”, “HiLyte Fluor680”, “IRDye700DX”, “methylene blue”, “Porphyrins”, “Porphysomes”, “VivoTag-680”, “VivoTag-S680”, “AlexaFluor750”, “AlexaFluor790”, “carbocyanine”, “conjugated copolymers”, “CW800-CA”, “Cy7”, “Cy7.5”, “cyanine dyes”, “Dy780”, “HiLyte Fluor750”, “Indocarbocyanine”, “IR-786”, “IRDye 800CW”, “IRDye 800RS”, “IRDye 800BK”, “Nervelight”, “OTL-38”, “Polymethine”, “VivoTag-S750”, “ASP5354”, and “Xanthene”.

Moreover, examples of the fluorescent substance derived from the observation target forming the observation target itself include “collagen”, “elastin”, and “NADH”.

Note that in the present embodiment, the light source device 3 is configured separately from the control device 9, but the present disclosure is not limited thereto, and a configuration in which the light source device 3 is provided inside the same housing as the control device 9 may be adopted.

The light guide 4 has the first end detachably connected to the light source device 3 and a second end detachably connected to the insertion unit 2. The light guide 4 propagates the first light and the second light supplied from the light source device 3 (the first light source 31 and the second light source 32) from the first end to the second end and supplies the light to the insertion unit 2. The first light and the second light supplied to the insertion unit 2 is emitted from a distal end of the insertion unit 2 and applied to the observation target. Return light (subject image) of the first light and the second light emitted from the observation target is condensed by the optical system in the insertion unit 2. The return light of the first light (hereinafter, described as first return light) includes, in addition to the first light reflected by the observation target, fluorescence emitted from a substance included in the observation target when the observation target is irradiated with the first light, and the substance is excited. In addition, the return light of the second light (hereinafter, described as second return light) is the second light reflected by the observation target.

The camera head 5 is detachably connected to a proximal end (an eyepiece unit 21 (FIG. 1)) of the insertion unit 2. Under the control of the control device 9, the camera head 5 captures the first return light and the second return light condensed by the insertion unit 2 to generate a pixel signal. Hereinafter, for convenience of description, the pixel signal generated through capturing of the first return light will be referred to as a fluorescence image. The fluorescence image corresponds to a first image according to the present disclosure. In addition, a pixel signal generated through capturing of the second return light will be referred to as a normal light image. The normal light image corresponds to a second image according to the present disclosure. In addition, the fluorescence image and the normal light image are collectively described as a captured image.

Note that a detailed configuration of the camera head 5 will be described in “Configuration of Camera Head” described later.

The first transmission cable 6 has a first end CN1 detachably connected to the control device 9 and a second end CN2 detachably connected to the camera head 5. Note that the second end CN2 is not limited to being detachably connected to the camera head 5 and may be fixed to the camera head 5. The first transmission cable 6 transmits a captured image output from the camera head 5 to the control device 9 and transmits a control signal, a synchronization signal, a clock, power, and the like transmitted from the control device 9 to the camera head 5.

Note that in the transmission of a captured image and the like from the camera head 5 to the control device 9 via the first transmission cable 6, the captured image and the like may be transmitted as an optical signal or may be transmitted as an electric signal. The same applies to transmission of a control signal, a synchronization signal, and a clock from the control device 9 to the camera head 5 via the first transmission cable 6.

The display device 7 is constituted by a display using liquid crystal, organic electro luminescence (EL), or the like and displays an image based on a video signal from the control device 9 under the control of the control device 9.

The second transmission cable 8 has a first end detachably connected to the display device 7 and a second end detachably connected to the control device 9. The second transmission cable 8 transmits a video signal processed by the control device 9 to the display device 7.

The control device 9 corresponds to a medical control device according to the present disclosure. The control device 9 includes a controller such as a central processing unit (CPU) or a micro processing unit (MPU) and integrally controls operations of the light source device 3, the camera head 5, and the display device 7. Note that the control device 9 is not limited to including the CPU or the MPU and may include an integrated circuit such as an application specific integrated circuit (ASIC), a field-programmable gate array (FPGA), or a graphics processing unit (GPU).

Note that a detailed configuration of the control device 9 will be described in “Configuration of Control Device” described later.

The third transmission cable 10 has a first end detachably connected to the light source device 3 and a second end detachably connected to the control device 9. The third transmission cable 10 transmits a control signal from the control device 9 to the light source device 3.

### Configuration of Camera Head

Next, the configuration of the camera head 5 will be described.

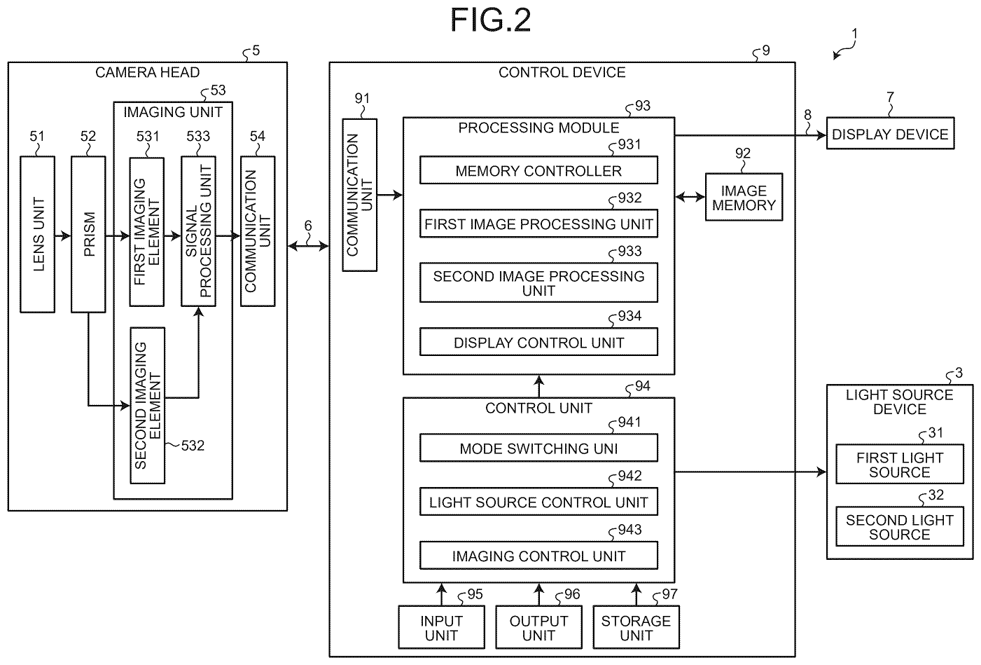

FIG. 2 is a block diagram illustrating configurations of the camera head 5 and the control device 9.

As illustrated in FIG. 2, the camera head 5 includes a lens unit 51, a prism 52, an imaging unit 53, and a communication unit 54.

The lens unit 51 includes one or a plurality of lenses. The lens unit 51 forms an image of the first return light (excitation light and fluorescence) condensed by the insertion unit 2 on an imaging surface of a first imaging element 531 (FIG. 2) and forms an image of the second return light (white light) condensed by the insertion unit 2 on an imaging surface of a second imaging element 532 (FIG. 2).

The prism 52 separates the first return light (excitation light and fluorescence) from the second return light (white light) via the lens unit 51. Then, the prism 52 causes the first return light (excitation light and fluorescence) to travel toward the first imaging element 531. Moreover, the prism 52 causes the second return light (white light) to travel toward the second imaging element 532.

The imaging unit 53 captures an image of an inside of a living body under the control of the control device 9. As illustrated in FIG. 2, the imaging unit 53 includes the first imaging element 531, the second imaging element 532, and a signal processing unit 533.

The first imaging element 531 and the second imaging element 532 receive the first return light and the second return light and convert the first return light and the second return light into electric signals (analog signals). In the present embodiment, each of the first imaging element 531 and the second imaging element 532 includes a complementary metal oxide semiconductor (CMOS) that is a rolling shutter type imaging element in which a plurality of pixels is arrayed in a two-dimensional shape in units of horizontal lines.

Here, although not specifically illustrated, the first imaging element 531 includes an invalid region that is not electrically guaranteed, an optical black region (OB region), and an effective pixel region that converts the first return light, an image of which is formed by the lens unit 51, into an imaging signal and outputs the imaging signal. Note that the second imaging element 532 similarly includes an invalid region, an optical black region (OB region), and an effective pixel region.

The first imaging element 531 captures an image of the first return light (excitation light and fluorescence) via the prism 52 under the control of the control device 9.

Note that an excitation light cut filter that removes only at least a part of the first light (excitation light) traveling toward the first imaging element 531 may be disposed on a front stage side of an optical path of the first imaging element 531.

In addition, the second imaging element 532 captures the second return light (white light) via the prism 52 under the control of the control device 9.

Note that the number of pixels of a fluorescence image and the number of pixels of a normal light image may be different from each other or may be the same.

Under the control of the control device 9, the signal processing unit 533 performs signal processing on captured images (analog signals) generated by the first imaging element 531 and the second imaging element 532 and outputs the captured images (digital signals).

For example, the signal processing unit 533 performs, on captured images (the analog signals) generated by the first imaging element 531 and the second imaging element 532, processing of removing reset noise, processing of multiplying an analog gain for amplifying the analog signals, and signal processing such as A/D conversion.

The communication unit 54 functions as a transmitter that transmits the captured images sequentially output from the imaging unit 53 to the control device 9 via the first transmission cable 6. The communication unit 54 is constituted by, for example, a high-speed serial interface that communicates the captured images with the control device 9 via the first transmission cable 6 at a transmission rate of 1 Gbps or more.

Note that the communication unit 54 may alternately or simultaneously transmit a fluorescence image and a normal light image to the control device 9.

### Configuration of Control Device

Next, the configuration of the control device 9 will be described with reference to FIG. 2.

As illustrated in FIG. 2, the control device 9 includes a communication unit 91, an image memory 92, a processing module 93, a control unit 94, an input unit 95, an output unit 96, and a storage unit 97.

The communication unit 91 functions as a receiver that receives the captured images sequentially transmitted from the camera head 5 (the communication unit 54) via the first transmission cable 6. The communication unit 91 is constituted by, for example, a high-speed serial interface that communicates the captured images with the communication unit 54 at a transmission rate of 1 Gbps or more.

The image memory 92 is constituted by, for example, a dynamic random access memory (DRAM) or the like. The image memory 92 may temporarily store a plurality of frames of the captured images sequentially output from the camera head 5 (the communication unit 54).

The processing module 93 processes the captured images sequentially transmitted from the camera head 5 (the communication unit 54) and received by the communication unit 91 under the control of the control unit 94. As illustrated in FIG. 2, the processing module 93 includes a memory controller 931, a first image processing unit 932, a second image processing unit 933, and a display control unit 934.

The memory controller 931 controls writing of a captured image into the image memory 92 and reading of the captured image from the image memory 92. More specifically, the memory controller 931 writes a fluorescence image received by the communication unit 91 into the image memory 92, reads the fluorescence image from the image memory 92 at a specific timing, and inputs the fluorescence image to the first image processing unit 932. In addition, the memory controller 931 writes a normal light image received by the communication unit 91 into the image memory 92, reads the normal light image from the image memory 92 at a specific timing, and inputs the normal light image to the second image processing unit 933.

The first image processing unit 932 executes first image processing on the input fluorescence image.

Examples of the first image processing include optical black subtraction processing, white balance adjustment processing, demosaic processing, color correction matrix processing, gamma correction processing, YC processing for converting an RGB signal into a luminance chrominance signal (Y, Cb/Cr signal), digital gain adjustment for multiplication by a digital gain, noise removal, and filter processing for enhancing a structure.

The second image processing unit 933 executes second image processing on the input normal light image.

Examples of the second image processing include optical black subtraction processing, white balance adjustment processing, demosaic processing, color correction matrix processing, gamma correction processing, YC processing for converting an RGB signal into a luminance chrominance signal (Y, Cb/Cr signal), digital gain adjustment, noise removal, and filter processing for enhancing a structure.

Note that the first image processing and second image processing may be image processing different from each other or may be the same image processing.

Under the control of the control unit 94, the display control unit 934 generates a video signal for displaying the fluorescence image after the first image processing is executed by the first image processing unit 932 and the normal light image after the second image processing is executed by the second image processing unit 933. Then, the display control unit 934 outputs the video signal to the display device 7 via the second transmission cable 8.

The control unit 94 is realized through executing of various programs stored in the storage unit 97 by a controller such as a CPU or a micro processing unit (MPU), controls operations of the light source device 3, the camera head 5, and the display device 7, and controls the entire operation of the control device 9. Note that the control unit 94 is not limited to including the CPU or the MPU and may be constituted by an integrated circuit such as an application specific integrated circuit (ASIC) or an FPGA. As illustrated in FIG. 2, the control unit 94 has functions as a mode switching unit 941, a light source control unit 942, and an imaging control unit 943.

The mode switching unit 941 switches a mode of the medical observation system 1 to either a normal observation mode or a fluorescence observation mode according to a user operation to the input unit 95.

The normal observation mode is a mode in which only a normal light image, of a fluorescence image and the normal light image, is generated, and the normal light image is displayed on the display device 7.

In the normal observation mode, the control unit 94 turns on only the second light source 32 of the first light source 31 and the second light source 32. In addition, the communication unit 54 sequentially transmits normal light images generated by the imaging unit 53 to the communication unit 91. Moreover, the processing module 93 executes the second image processing on each of the normal light images received by the communication unit 91, generates a video signal for displaying the normal light image after execution of the second image processing, and outputs the video signal to the display device 7. As a result, the normal light image is displayed on the display device 7.

The fluorescence observation mode is a mode in which a fluorescence image and a normal light image are generated, and the fluorescence image and the normal light image (or a superimposed image obtained by superimposing the fluorescence image and the normal light image) are displayed on the display device 7.

In the fluorescence observation mode, the control unit 94 simultaneously turns on the first light source 31 and the second light source 32. In addition, the communication unit 54 transmits the fluorescence image and the normal light image generated by the imaging unit 53 to the communication unit 91. Moreover, the processing module 93 executes the image processing on each of the fluorescence image and the normal light image received by the communication unit 91, generates a video signal for displaying the fluorescence image and the normal light image after execution of the image processing (or a superimposed image obtained by superimposing the fluorescence image and the normal light image), and outputs the video signal to the display device 7. As a result, the fluorescence image and the normal light image (or the superimposed image obtained by superimposing the fluorescence image and the normal light image) are displayed on the display device 7.

Note that functions of the light source control unit 942 and the imaging control unit 943 will be described in “Problem” and “Gain Control, Light Source Control, and Electronic Shutter Control” described later.

The input unit 95 corresponds to an operation receiving unit according to the present disclosure. The input unit 95 is composed using an operation device such as a mouse, a keyboard, and a touch panel and receives a user operation by a user such as an operator. The input unit 95 outputs an operation signal corresponding to the user operation to the control unit 94. Note that the operation receiving unit according to the present disclosure is not limited to a configuration in which the operation receiving unit is provided in the control device 9 like the input unit 95 and may be a configuration in which the operation receiving unit is provided in the camera head 5, for example.

The output unit 96 is composed using a speaker, a printer, or the like and outputs various types of information.

The storage unit 97 stores a program executed by the control unit 94, information necessary for processing of the control unit 94, and the like.

By the way, in general, in the medical observation system 1, it is difficult to adjust a signal value related to the fluorescence image generated by the first imaging element 531 that has received the first return light (excitation light and fluorescence) due to influence of the following (1) to (4).

### (1) Agent

In general, a light amount of fluorescence varies depending on the type of agent and the dose thereof.

The type of agent to be administered to the observation target is selected according to the observation target (cancer, blood, lymph, or the like.). Moreover, in order to capture fluorescence generated from the agent in the medical observation system 1, an agent capable of separating a wavelength of excitation light for exciting the agent from a wavelength of the fluorescence generated from the agent is selected. Agents selected in this manner have different light amounts of fluorescence.

In addition, although the light amount of fluoresce may be adjusted by the dose of an agent, it is difficult to increase the dose more than necessary in order to realize a minimally invasive treatment.

That is, it is difficult to adjust the light amount of fluorescence by selecting the type of agent and adjusting the dose thereof, and as a result, it is difficult to adjust the signal value related to the fluorescence image generated by the first imaging element 531 of the medical observation system 1 by selecting the type of agent and adjusting the dose thereof.

### (2) Observation Target

The light amount of fluorescence varies depending on the site and state of the observation target.

Specifically, in a site and state in which an agent is likely to stagnate, the light amount of fluorescence increases. On the other hand, in a site and state in which the agent easily flows and hardly stagnates, the light amount of fluorescence decreases, and light remaining time also decreases. In addition, when the observation target is a tumor, the light amount of fluorescence received by the first imaging element 531 of the medical observation system 1 changes depending on the spread, size, and depth of the tumor.

That is, it is difficult to adjust the light amount of fluorescence according to the type and situation of the observation target, and as a result, it is difficult to adjust the signal value related to the fluorescence image generated by the first imaging element 531 of the medical observation system 1 according to the type and situation of the observation target.

### (3) Light Source Device

The light amount of fluorescence varies depending on the light amount of the first light (excitation light) emitted from the light source device 3.

In order to increase the light amount of the excitation light, it may be necessary to adjust power supplied to the light source device 3, but the power that may be supplied to the light source device 3 is limited according to an upper limit value of the power for operating the entire medical observation system 1. In addition, it is required to adjust the light amount of the excitation light in consideration of heat generation (for example, heat generation between the light guide 4 and the insertion unit 2, or the like) in a member constituting an optical path of the excitation light, correspondence to a laser class, an amount of light energy received by the observation target or the living body in the vicinity (there is a risk of causing a burn if the amount of light energy is large), a fading rate of agent fluorescence, or the like. Moreover, the adjustment of the light amount of the excitation light may be realized by changing the number of light sources of the excitation light mounted on the light source device 3, but the number of light sources affects the size of the light source device 3, and the size may be limited by the size of a cart for carrying the light source device 3 or the like.

That is, it is difficult to adjust the light amount of fluorescence by adjusting the light amount of the excitation light, and as a result, it is difficult to adjust the signal value related to the fluorescence image generated by the first imaging element 531 of the medical observation system 1 by adjusting the light amount of the excitation light.

### (4) Imaging Element

Depending on the light amount of fluorescence received by the first imaging element 531, the signal value related to the fluorescence image generated by the first imaging element 531 is different.

In order to adjust the signal value related to the fluorescence image, it is desired to select the first imaging element 531 having optimum sensitivity, configuration, and the like for capturing fluorescence. However, the first imaging element 531 may be required not only to output an image for fluorescence observation based on reception of fluorescence but also to output an image for normal light observation based on reception of visible light such as white light. In that case, the first imaging element 531 needs to select an element capable of corresponding to both fluorescence observation and normal light observation, and in some cases, an imaging element having optimum characteristics for capturing fluorescence may not be used. In addition, the first imaging element 531 is disposed in the camera head 5, but since the camera head 5 has a size and weight suitable for observation, the type of the first imaging element 531 including the size may be limited.

That is, it is difficult to adjust the signal value related to the fluorescence image generated by the first imaging element 531 of the medical observation system 1 by selecting the type of the first imaging element 531.

As described above, since the signal value related to the fluorescence image generated by the first imaging element 531 is determined under the above constraints, adjustment is not easy.

### Problem

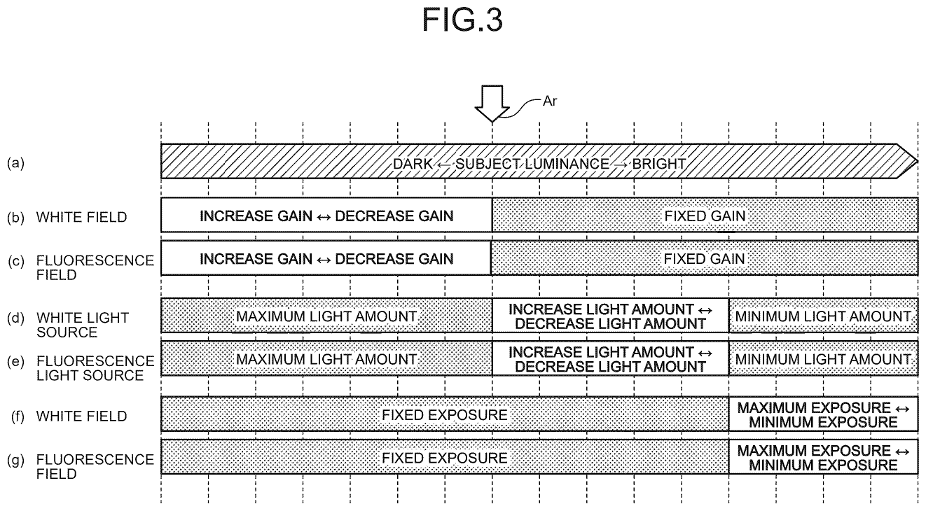

FIG. 3 is a diagram for explaining a problem. (a) of FIG. 3 illustrates subject luminance (luminance level) of a normal light image. In FIG. 3, the subject luminance increases toward a right side in the drawing, and the subject luminance decreases toward a left side in the drawing. (b) of FIG. 3 and (c) of FIG. 3 illustrate gain control (adjustment of analog gain and digital gain), which is signal processing. More specifically, (b) of FIG. 3 illustrates gain control in a normal light image. In addition, (c) of FIG. 3 illustrates gain control in a fluorescence image. (d) of FIG. 3 and (e) of FIG. 3 illustrate control of light amounts (light source control (current dimming)) of the first light and the second light to the observation target in the light source device 3. More specifically, (d) of FIG. 3 illustrates light source control (current dimming) of the second light source 32. (e) of FIG. 3 illustrates light source control (current dimming) of the first light source 31. (f) of FIG. 3 and (g) of FIG. 3 illustrate electronic shutter control in the first imaging element 531 and the second imaging element 532. More specifically, (f) of FIG. 3 illustrates electronic shutter control in the second imaging element 532. (g) of FIG. 3 illustrates electronic shutter control in the first imaging element 531.

The control unit 94 calculates subject luminance (luminance average value) in a detection area, which is at least a part of the entire image area of the normal light image, based on a luminance signal (Y signal), in the detection area, of a luminance chrominance signal (Y, Cb/Cr signal), which is the normal light image subjected to the YC processing in the first image processing unit 932 or the second image processing unit 933. Then, the control unit 94 executes dimming control for adjusting the captured image to reference brightness based on the calculated subject luminance.

Specifically, as illustrated in (b) of FIG. 3 and (c) of FIG. 3, when the calculated subject luminance is darker than the subject luminance (subject luminance indicated by an arrow Ar in FIG. 3) serving as a specific reference, the control unit 94 brightens the subject luminance by gain control (adjustment of an analog gain and a digital gain by the imaging control unit (gain control unit) 943) as signal processing. In addition, as illustrated in (d) of FIG. 3 and (e) of FIG. 3, when the calculated subject luminance is brighter than the subject luminance serving as a specific reference, the control unit 94 first decreases light amounts of the first light and the second light emitted from the first light source 31 and the second light source 32 by decreasing current values supplied to the first light source 31 and the second light source 32 (light source control (current dimming) by the light source control unit 942), and then decreases an opening amount of an electronic shutter in the first imaging element 531 and an opening amount of an electronic shutter in the second imaging element 532 (electronic shutter control by the imaging control unit (shutter control unit) 943).

Here, in the known gain control, light source control (current dimming), and electronic shutter control, the same gains (analog gain and digital gain) are multiplied in a white field (field for generating a normal light image) and a fluorescence field (field for generating a fluorescence image) to adjust the light amounts of the first light and the second light at the same ratio, and further, the opening amount of the electronic shutter in the first imaging element 531 and the opening amount of the electronic shutter in the second imaging element 532 are adjusted at the same ratio.

By the way, as described above, there are various types of agents to be administered to the observation target such as an agent emitting bright fluorescence and an agent emitting only dark fluorescence depending on the type of the agent. In addition, there are various sites such as a site where an agent is deposited to emit light brightly and a site where concentration of the agent is low due to reflux or metabolism of the observation target, and thus the light amount of fluorescence becomes weak. Therefore, a function of adjusting brightness is required from a user who performs fluorescence observation. This function is generally realized basically by gain control, but when a high-sensitivity imager is used as the first imaging element 531, the image may become bright without multiplication of gains (analog gain and digital gain). In this case, a negative gain is multiplied in the signal processing, but if the signal value related to the fluorescence image is saturated, there is a problem in which gradation of the signal value may not be restored.

In the present embodiment, in order to cope with the above-described problem, the electronic shutter control is mainly set to be different from the known one.

Note that details of the electronic shutter control will be described in “Gain Control, Light Source Control, and Electronic Shutter Control”.

### Gain Control, Light Source Control, and Electronic Shutter Control

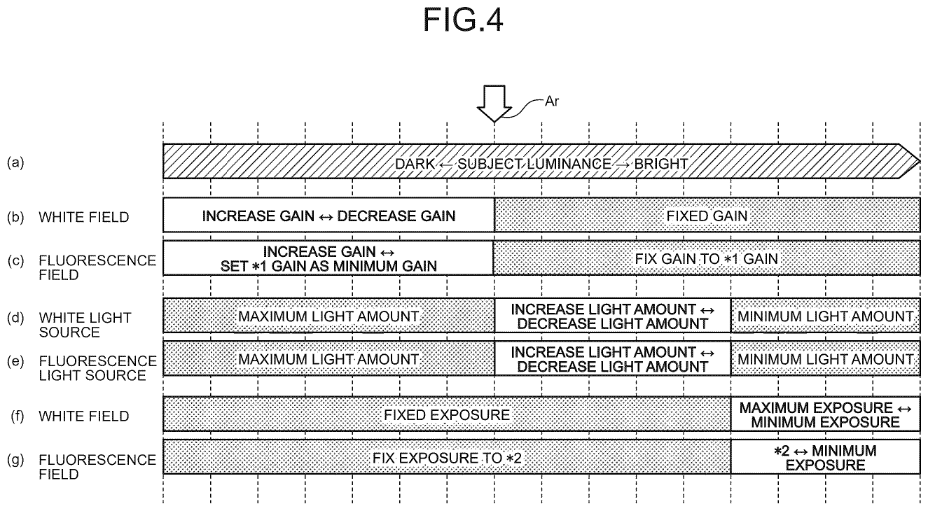

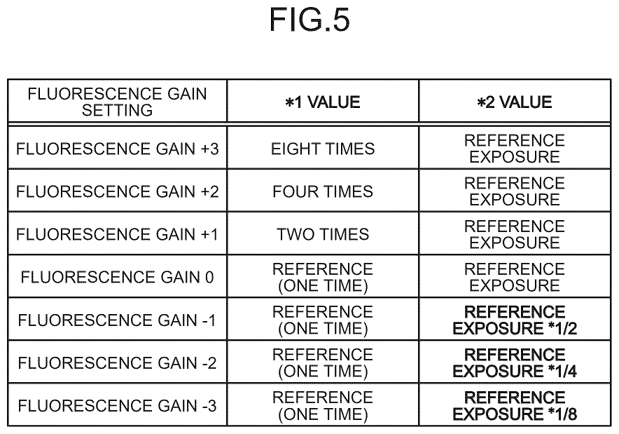

FIG. 4 is a diagram for explaining gain control, light source control, and electronic shutter control according to the embodiment. Specifically, (a) to (g) of FIG. 4 correspond to (a) to (g) of FIG. 3, respectively. FIG. 5 is a table illustrating fluorescence gains set by a user.

The control unit 94 calculates subject luminance (luminance average value) in a detection area, which is at least a part of the entire image area of the normal light image, based on a luminance signal (Y signal), in the detection area, of a luminance chrominance signal (Y, Cb/Cr signal), which is the normal light image subjected to the YC processing in the first image processing unit 932 or the second image processing unit 933. Then, the control unit 94 executes dimming control for adjusting the captured image to reference brightness based on the calculated subject luminance.

Specifically, as illustrated in (b) of FIG. 4 and (c) of FIG. 4, when the calculated subject luminance is darker than the subject luminance (subject luminance indicated by the arrow Ar in FIG. 4) serving as a specific reference, the control unit 94 brightens the subject luminance by gain control (adjustment of an analog gain and a digital gain by the imaging control unit (gain control unit) 943) as signal processing. In addition, as illustrated in (d) of FIG. 4 and (e) of FIG. 4, when the calculated subject luminance is brighter than the subject luminance serving as a specific reference, the control unit 94 first decreases light amounts of the first light and the second light emitted from the first light source 31 and the second light source 32 by decreasing current values supplied to the first light source 31 and the second light source 32 (light source control (current dimming) by the light source control unit 942), and then decreases an opening amount of an electronic shutter in the first imaging element 531 and an opening amount of an electronic shutter in the second imaging element 532 (electronic shutter control by the imaging control unit (shutter control unit) 943).

Here, in the gain control in the present embodiment, the same gains (analog gain and digital gain) are not multiplied in the white field and the fluorescence field. More specifically, the imaging control unit (gain control unit) 943 multiplies gains (analog gain and digital gain) for the white field as in the related art (see “Problem” described above). On the other hand, for the fluorescence field, the imaging control unit (gain control unit) 943 sets gains (analog gain and digital gain) corresponding to a fluorescence gain (FIG. 5) selected by the user operation on the input unit 95 as minimum values and adjusts the gains. As illustrated in FIG. 5, the fluorescence gain is information in which gains (analog gain and digital gain) and the opening amount of the electronic shutter in the first imaging element 531 are associated with each other according to numerical values (+3 to −3) and is stored in the storage unit 97. For example, when the fluorescence gain+2 is selected by the user operation, the imaging control unit (gain control unit) 943 sets gains (analog gain and digital gain) that are four times the reference gains (analog gain and digital gain) as minimum values and adjusts the gains.

In addition, in the light source control (current dimming) in the present embodiment, the light amounts of the first light and the second light are adjusted at the same ratio as in the related art (see “Problem” described above).

Moreover, in the electronic shutter control in the present embodiment, the opening amount of the electronic shutter in the first imaging element 531 and the opening amount of the electronic shutter in the second imaging element 532 are not adjusted at the same ratio. More specifically, for the white field, the imaging control unit (shutter control unit) 943 adjusts the opening amount of the electronic shutter in the second imaging element 532 as in the related art (see “Problem” described above). On the other hand, for the fluorescence field, the imaging control unit (shutter control unit) 943 sets the opening amount according to a fluorescence gain (FIG. 5) selected by the user operation on the input unit 95 as a maximum value and adjusts the opening amount of the electronic shutter in the first imaging element 531. For example, when the fluorescence gain −2 is selected by the user operation, the imaging control unit (shutter control unit) 943 sets ¼ of the reference opening amount (reference exposure illustrated in FIG. 5) as a maximum value and adjusts the opening amount of the electronic shutter in the first imaging element 531.

That is, in the present embodiment, when a first ratio is a ratio of a signal value related to a fluorescence image to a signal value related to a normal light image in a case where an opening amount of an electronic shutter in the first imaging element 531 is a first opening amount (for example, a reference opening amount (reference exposure illustrated in FIG. 5)), the imaging control unit (shutter control unit) 943 changes the opening amount of the electronic shutter in the first imaging element 531 from the first opening amount to a second opening amount smaller than the first opening amount (for example, ¼ of the reference opening amount (reference exposure illustrated in FIG. 5)), thereby setting a ratio of the signal value related to the fluorescence image to the signal value related to the normal light image to a second ratio smaller than the first ratio.

In addition, in the present embodiment, the imaging control unit (gain control unit) 943 uses gains larger than gain reference values (reference illustrated in FIG. 5) as gains (analog gain and digital gain) when the brightness of the fluorescence image is darker than the reference brightness (subject luminance indicated by the arrow Ar in FIG. 4). Moreover, when the brightness of the fluorescence image is brighter than the reference brightness, the imaging control unit (shutter control unit) 943 sets the opening amount of the electronic shutter in the first imaging element 531 to an opening amount smaller than an opening amount reference value (reference exposure illustrated in FIG. 5).

Moreover, in the present embodiment, the first return light is first fluorescence based on a first fluorescence agent. The imaging control unit (shutter control unit) 943 is configured to be able to set the opening amount of the electronic shutter to the first opening amount when the first return light is captured and set the opening amount of the electronic shutter to the second opening amount when second fluorescence based on a second fluorescence agent emitting fluorescence weaker than the first fluorescence is captured.

According to the present embodiment described above, the following effects are obtained.

In the control device 9 according to the present embodiment, when a first ratio is a ratio of a signal value related to a fluorescence image to a signal value related to a normal light image in a case where an opening amount of an electronic shutter in the first imaging element 531 is a first opening amount (for example, a reference opening amount (reference exposure illustrated in FIG. 5)), the imaging control unit (shutter control unit) 943 changes the opening amount of the electronic shutter in the first imaging element 531 from the first opening amount to a second opening amount smaller than the first opening amount (for example, ¼ of the reference opening amount (reference exposure illustrated in FIG. 5)), thereby setting a ratio of the signal value related to the fluorescence image to the signal value related to the normal light image to a second ratio smaller than the first ratio.

Therefore, when the signal value related to the fluorescence image is saturated without using the electronic shutter, for example, even in a case where an agent that emits bright fluorescence is used, the brightness of the fluorescence image may be adjusted to appropriate brightness without losing the gradation of the fluorescence image.

Therefore, the control device 9 according to the present embodiment may generate a fluorescence image suitable for observation.

### OTHER EMBODIMENTS

Although the embodiment for carrying out the present disclosure has been described so far, the present disclosure should not be limited only by the above-described embodiment.

In the above-described embodiment, a configuration capable of executing only the fluorescence observation mode may be adopted. That is, the second imaging element 532, the second light source 32, and the second image processing unit 933 may be omitted. In such a configuration, when the signal value related to the fluorescence image is saturated in a case where the opening amount of the electronic shutter in the first imaging element 531 is set to the first opening amount, the imaging control unit (shutter control unit) 943 sets the opening amount to the second opening amount smaller than the first opening amount to desaturate the signal value.

In the above-described embodiment, the number of fluorescence observation modes is not limited to one, and for example, a plurality of fluorescence observation modes may be provided for each type of agent to be administered to the observation target. At this time, the fluorescence gain (FIG. 5) stored in the storage unit 97 is also prepared for each fluorescence observation mode. That is, the imaging control unit (shutter control unit) 943 refers to the fluorescence gain according to the fluorescence observation mode switched by the mode switching unit 941 and changes the opening amount of the electronic shutter in the first imaging element 531 according to the fluorescence gain.

In the above-described embodiment, the imaging control unit (shutter control unit) 943 controls the electronic shutter in the first imaging element 531, but the present disclosure is not limited thereto, and a configuration in which a mechanical shutter corresponding to the first imaging element 531 is controlled may be adopted.

In the above-described embodiment, first to fourth modifications described below may be adopted.

### First Modification

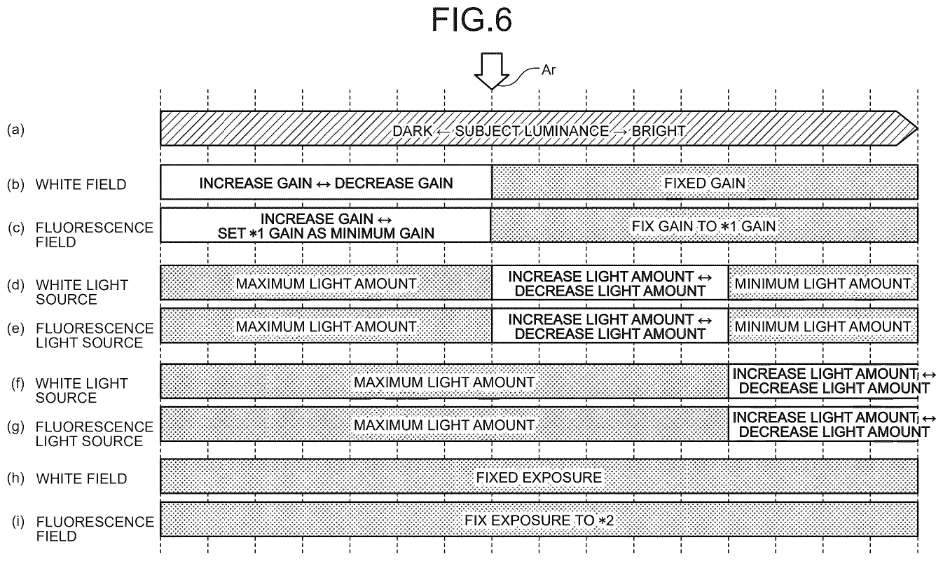

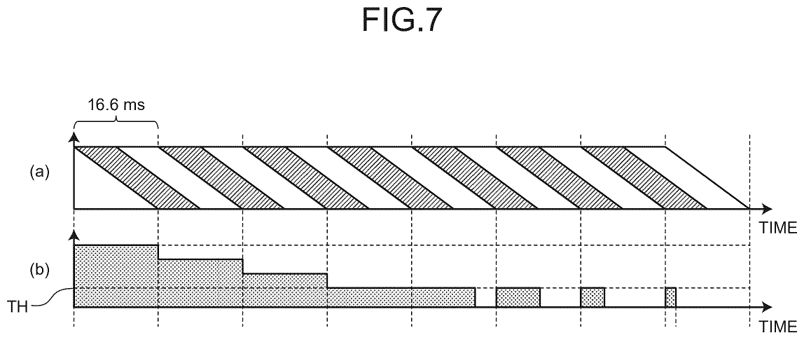

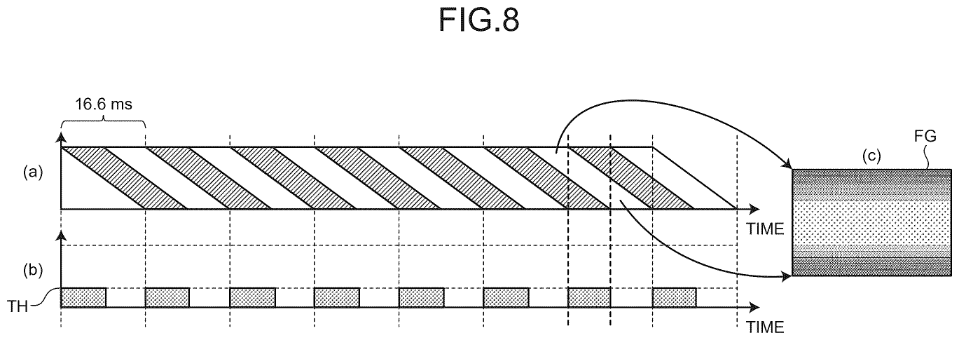

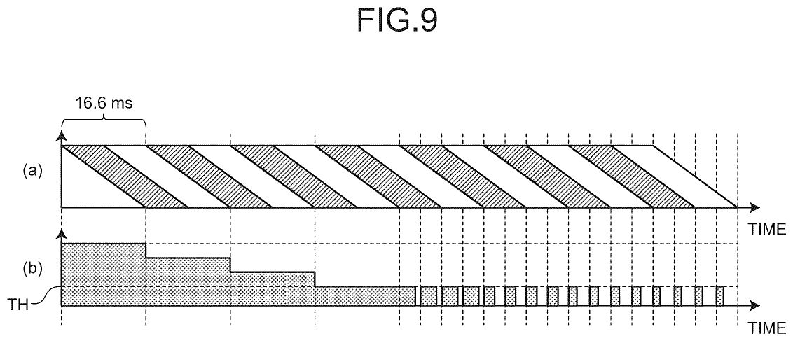

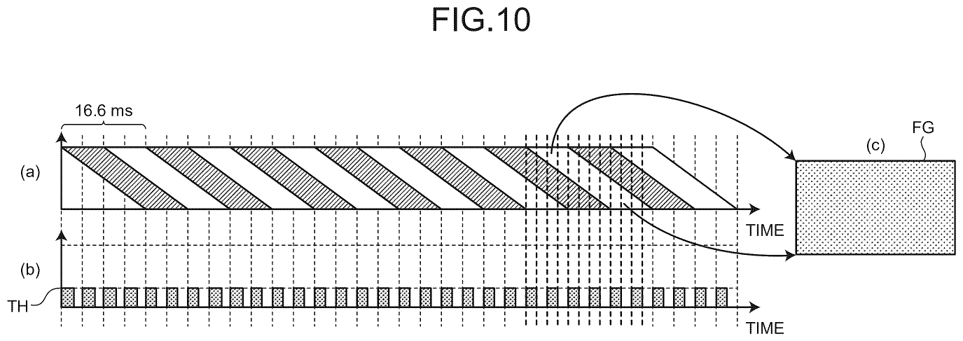

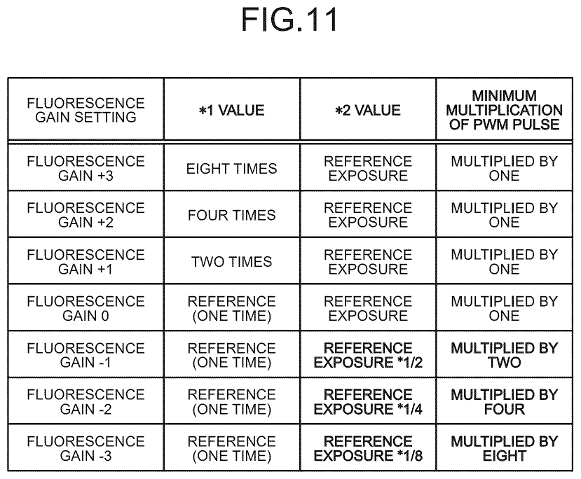

FIGS. 6 to 11 are diagrams and a table for explaining the first modification of the embodiment. Specifically, (a) to (e) of FIG. 6 correspond to (a) to (e) of FIG. 4, respectively. (f) of FIG. 6 and (g) of FIG. 6 illustrate control of light amounts of the first light and the second light to the observation target in the light source device 3 (light source control (pulse width modulation (PWM) dimming)). More specifically, (f) of FIG. 6 illustrates light source control (PWM dimming) of the second light source 32. (g) of FIG. 6 illustrates light source control (PWM dimming) of the first light source 31. (h) of FIG. 6 and (i) of FIG. 6 correspond to (f) of FIG. 4 and (g) of FIG. 4, respectively. FIGS. 7 and 8 are diagrams for explaining problems in a case of using PWM dimming. More specifically, in (a) of FIG. 7 and (a) of FIG. 8, a vertical axis represents a horizontal line of the first imaging element 531 (an uppermost stage represents an uppermost horizontal line (a first horizontal line), a lowermost stage represents a lowermost horizontal line (a last line)), and a horizontal axis represents time. A parallelogram region is a region that contributes to generation of a fluorescence image in one field. In (b) of FIG. 7 and (b) of FIG. 8, a vertical axis represents a current value supplied to the first light source 31, and a horizontal axis represents time (supply time of a current supplied to the first light source 31). (c) of FIG. 8 is a diagram schematically illustrating a generated fluorescence image FG. FIGS. 9 and 10 are diagrams for explaining control for solving the problems in the case of using PWM dimming. More specifically, (a) of FIG. 9 and (a) of FIG. 10 correspond to (a) of FIG. 7 and (a) of FIG. 8, respectively. (b) of FIG. 9 and (b) of FIG. 10 correspond to (b) of FIG. 7 and (b) of FIG. 8, respectively. (c) of FIG. 10 corresponds to (c) of FIG. 8. FIG. 11 is a table corresponding to FIG. 5 and illustrating fluorescence gains set by the user.

In the above-described embodiment, as a method for reducing brightness of a captured image in a time direction, PWM dimming for adjusting light emission time of the first light and the second light emitted from the first light source 31 and the second light source 32 may be adopted.

Specifically, as illustrated in (b) of FIG. 6 and (c) of FIG. 6, when calculated subject luminance is darker than subject luminance (subject luminance indicated by the arrow Ar in FIG. 6) serving as a specific reference, the imaging control unit (gain control unit) 943 brightens the subject luminance by gain control (adjustment of an analog gain and a digital gain) which is signal processing, as in the above-described embodiment.

In addition, as illustrated in (d) of FIG. 6, (e) of FIG. 6, (b) of FIG. 7, and (b) of FIG. 9, when the calculated subject luminance is brighter than the subject luminance serving as a specific reference, the light source control unit 942 decreases the light amounts of the first light and the second light emitted from the first light source 31 and the second light source 32 by decreasing the current values supplied to the first light source 31 and the second light source 32 (current dimming), as in the above-described embodiment.

Then, as illustrated in (f) of FIG. 6, (g) of FIG. 6, (b) of FIG. 7, and (b) of FIG. 9, when the subject luminance is further darkened after the current values supplied to the first light source 31 and the second light source 32 are lowered to a drive limit value TH, the light source control unit 942 decreases supply time of currents supplied to the first light source 31 and the second light source 32 and decreases light emission time of the first light and the second light (PWM dimming).

Note that, in electronic shutter control in the first modification, as illustrated in (h) of FIG. 6 and (i) of FIG. 6, the opening amount of the electronic shutter in the first imaging element 531 and the opening amount of the electronic shutter in the second imaging element 532 are fixed. More specifically, the opening amount of the electronic shutter in the first imaging element 531 is fixed to an opening amount of the electronic shutter according to the fluorescence gain (FIG. 11) selected by the user operation on the input unit 95. As illustrated in FIG. 11, the fluorescence gain is information in which gains (analog gain and digital gain), the opening amount of the electronic shutter in the first imaging element 531, and minimum multiplication of a PWM pulse (first light) are associated with each other according to numerical values (+3 to −3) and is stored in the storage unit 97. For example, when the fluorescence gain −1 is selected by the user operation, the opening amount of the electronic shutter in the first imaging element 531 is fixed to ½ of the reference opening amount (reference exposure illustrated in FIG. 11).

Here, for example, as illustrated in (a) of FIG. 7 and (a) of FIG. 8, when PWM dimming is performed in a state where the opening amount of the electronic shutter in the first imaging element 531 is fixed to ½ of the reference opening amount (reference exposure illustrated in FIG. 11), there are the following problems. Note that, in (a) of FIG. 7 and (a) of FIG. 8, a hatched parallelogram region is a region indicating sweeping and discarding of charges by the electronic shutter. A non-hatched parallelogram region is a region indicating an effective exposure period.

Specifically, as illustrated in FIG. 8, when the light emission time of the first light is reduced by PWM dimming, the first light does not strike horizontal lines on an upper side and a lower side of the first imaging element 531 in the region (parallelogram region not shaded) indicating the effective exposure period. Therefore, as illustrated in (c) of FIG. 8, there is a problem in which the generated fluorescence image FG becomes dark in upper and lower portions and becomes an image having unevenness in brightness.

Therefore, in the first modification, as illustrated in FIGS. 9 and 10, when PWM dimming is performed, the light source control unit 942 intermittently turns on the first light emitted from the first light source 31 for each fluorescence field at a predetermined cycle obtained by dividing a period of the fluorescence field. As a result, as illustrated in FIG. 10, the first light strikes the entire region (parallelogram region not shaded) indicating the effective exposure period, and unevenness in brightness does not occur in the generated fluorescence image FG ((c) of FIG. 10). Note that FIGS. 9 and 10 illustrate a case of multiplication by four through dividing of the period of the fluorescence field into four. As the multiplication, multiplication corresponding to a fluorescence gain (FIG. 11) selected by the user operation is adopted. The minimum multiplication of the PWM pulse illustrated in FIG. 11 is merely an example of the minimum multiplication, and values larger than the values of the multiplication described above may be adopted.

### Second Modification

A medical observation system according to the second modification is a medical observation system using a so-called video scope (flexible endoscope) having an imaging unit on a distal end side of an insertion unit. Hereinafter, for convenience of description, the medical observation system 1 according to the second modification will be referred to as a medical observation system 1B.

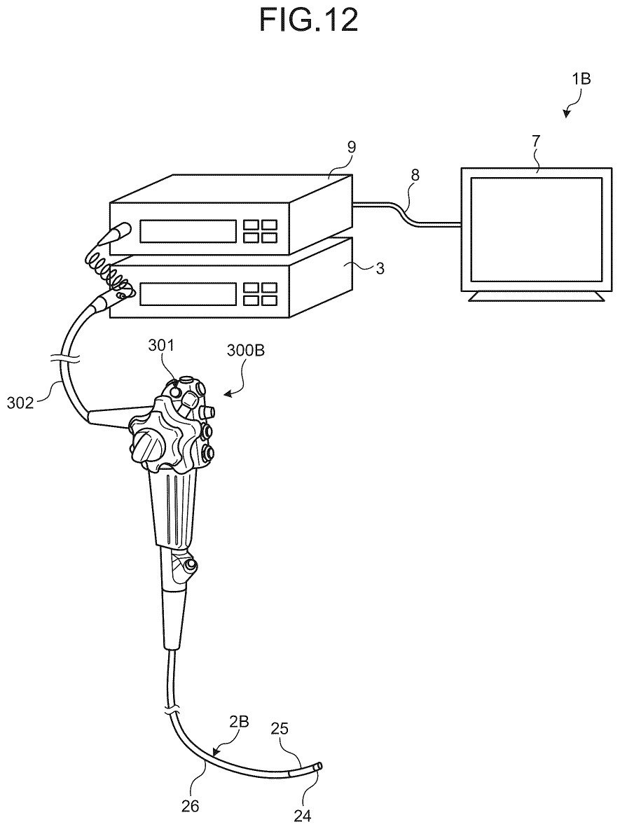

FIG. 12 is a view for explaining the second modification of the embodiment.

As illustrated in FIG. 12, the medical observation system 1B includes an endoscope 300B that captures an in-vivo image of an observed region by inserting an insertion unit 2B into a living body and outputs a captured image, the light source device 3 that emits the first light and the second light from a distal end of the endoscope 300B, the control device 9 that processes the captured image output from the endoscope 300B, and the display device 7 that is connected to the control device 9 via the second transmission cable 8 and displays an image based on a video signal processed by the control device 9.

As illustrated in FIG. 12, the endoscope 300B includes the insertion unit 2B having a flexible elongated shape, an operation unit 301 connected to a proximal end side of the insertion unit 2B and receiving various operations, and a universal cord 302 extending in a direction different from a direction in which the insertion unit 2B extends from the operation unit 301 and incorporating various cables connected to the light source device 3 and the control device 9.

As illustrated in FIG. 12, the insertion unit 2B includes a distal end portion 24, a bendable bending portion 25 connected to a proximal end side of the distal end portion 24 and composed of a plurality of bending pieces, and an elongated flexible tube portion 26 connected to a proximal end side of the bending portion 25 and having flexibility.

Although not specifically illustrated, substantially the same configuration as the camera head 5 described in the above-described embodiment is incorporated in the distal end portion 24. A captured image captured by the distal end portion 24 (the first imaging element 531 and the second imaging element 532) is output to the control device 9 via the operation unit 301 and the universal cord 302.

Even when the configuration of the second modification described above is adopted, the same effects as those of the above-described embodiment are obtained.

### Third Modification

A medical observation system according to the third modification is a medical observation system using a surgical microscope that enlarges and captures a predetermined field of view of an inside of a subject (inside of a living body) or a surface of the subject (surface of a living body) to be observed. Hereinafter, for convenience of description, the medical observation system 1 according to the third modification will be referred to as a medical observation system 1C.

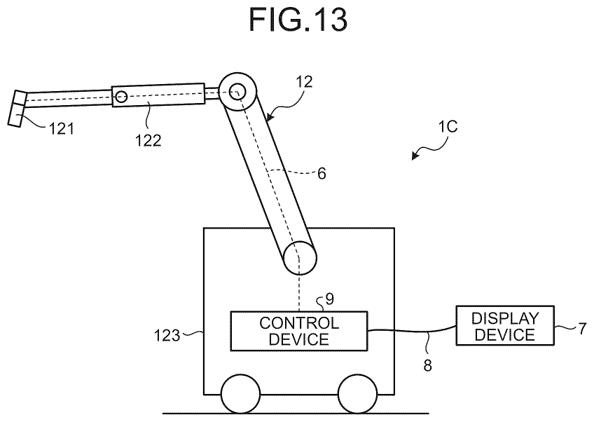

FIG. 13 is a diagram for explaining the third modification of the embodiment.

As illustrated in FIG. 13, the medical observation system 1C includes a surgical microscope 12 that captures an image for observing a subject and outputs a captured image, the control device 9 that processes the captured image output from the surgical microscope 12, and the display device 7 that is connected to the control device 9 via the second transmission cable 8 and displays an image based on a video signal processed by the control device 9.

As illustrated in FIG. 13, the surgical microscope 12 includes a microscope unit 121 that enlarges a minute site of a subject, captures an image of the minute site, and outputs the captured image, a support unit 122 that is connected to a proximal end portion of the microscope unit 121 and includes an arm that rotatably supports the microscope unit 121, and a base unit 123 that rotatably holds a proximal end portion of the support unit 122 and is movable on a floor surface.

In addition, as illustrated in FIG. 13, the control device 9 is installed in the base unit 123. Moreover, although not specifically illustrated, the base unit 123 is also provided with the light source device 3 that emits the first light and the second light from the surgical microscope 12 to an observation target.

Note that the base unit 123 may be fixed to a ceiling, a wall surface, or the like to support the support unit 122, instead of being movably provided on the floor surface.

Although not specifically illustrated, substantially the same configuration as the camera head 5 described in the above-described embodiment is incorporated in the microscope unit 121. A captured image captured by the microscope unit 121 (the first imaging element 531 and the second imaging element 532) is output to the control device 9 via the first transmission cable 6 wired along the support unit 122.

Even when the configuration of the third modification described above is adopted, the same effects as those of the above-described embodiment are obtained.

### Fourth Modification

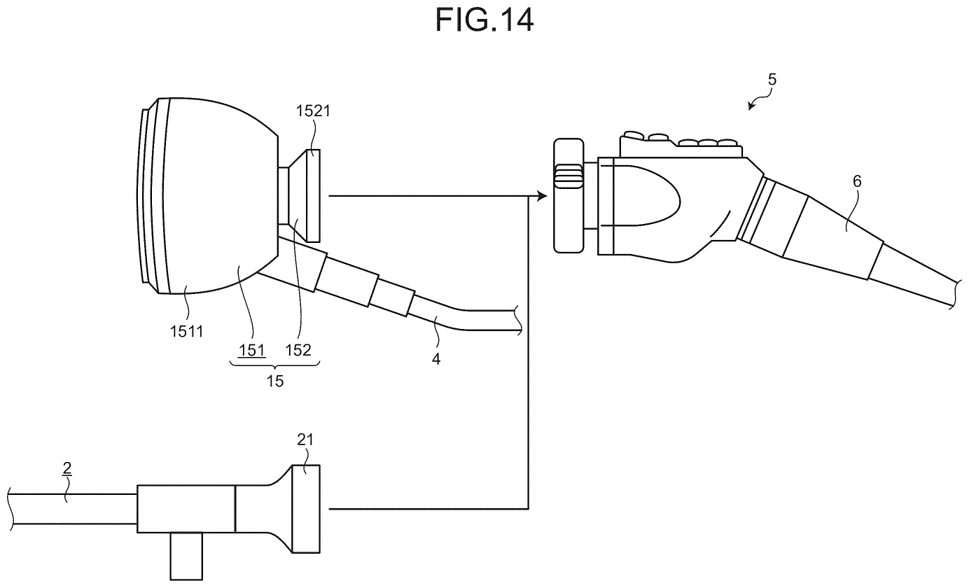

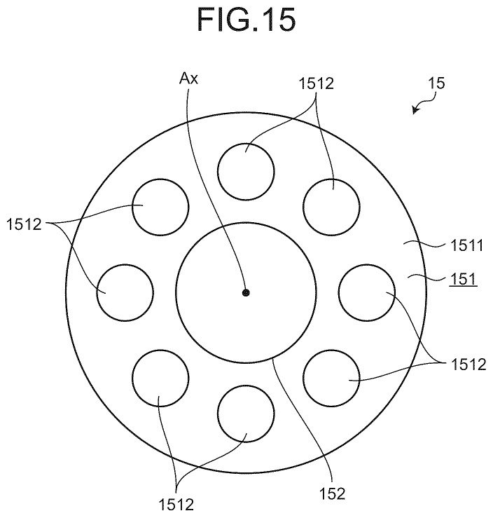

FIGS. 14 and 15 are views for explaining the fourth modification of the embodiment. Specifically, FIG. 14 is a side view of a ring light 15. FIG. 15 is a view of the ring light 15 as viewed from a front side (left side in FIG. 14).

In the fourth modification, in addition to the insertion unit 2 described in the above-described embodiment, the ring light 15 illustrated in FIGS. 14 and 15 is detachably connected to the camera head 5. That is, as illustrated in FIG. 14, the insertion unit 2 may be connected to the camera head 5, or the ring light 15 may be connected to the camera head 5 depending on a use state of the user.

The ring light 15 is not inserted into the observation target like the insertion unit 2, but supplies the first light and the second light to a surgical site and takes in return light of the first light and the second light from the surgical site. As illustrated in FIGS. 14 and 15, the ring light 15 includes an illumination unit 151 and a subject image capturing unit 152 that captures a subject image.

As illustrated in FIGS. 14 and 15, the illumination unit 151 includes a housing 1511 and a plurality of illumination lenses 1512.

The housing 1511 has an annular shape centered on an optical axis Ax. The second end of the light guide 4 is detachably connected to the housing 1511.

As illustrated in FIG. 15, the plurality of illumination lenses 1512 is arranged at predetermined intervals in a circumferential direction centered on the optical axis Ax on an end surface on a front side of the housing 1511. The plurality of illumination lenses 1512 irradiates the surgical site with the first light and the second light supplied from the light source device 3 and introduced into the housing 1511 via the light guide 4.

The subject image capturing unit 152 extends along the optical axis Ax. In addition, an inside of the subject image capturing unit 152 is provided with an optical system composed of one or a plurality of lenses and condenses return light of the first light and the second light emitted from the plurality of illumination lenses 1512 and passing through the surgical site. Moreover, a connecting portion 1521 is provided at an end portion on a proximal end side (right side in FIG. 14) of the subject image capturing unit 152. The connecting portion 1521 is designed (shaped) to be compatible with the eyepiece unit 21 in the insertion unit 2 and is detachably connected to the camera head 5.

Even when the configuration of the fourth modification described above is adopted, the same effects as those of the above-described embodiment are obtained.

Note that the following configurations also belong to the technical scope of the present disclosure.

The medical control device and the medical observation system according to the present disclosure are capable of generating an image suitable for observation.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical control device comprising:
an imaging control unit configured to
control an operation of an imaging element, and
cause the imaging element to capture first return light from an observation target irradiated with first light that is narrow band light and second return light from the observation target irradiated with second light having a wavelength band different from a wavelength band of the narrow band light, wherein
the imaging control unit includes a shutter control unit configured to change a signal value related to a first image by controlling an opening amount of a shutter for imaging, the first image being generated by the imaging element when the first return light is captured, and
the shutter control unit is configured to change the opening amount of the shutter from a first opening amount to a second opening amount smaller than the first opening amount to set a ratio of the signal value related to the first image to a signal value related to a second image to a second ratio smaller than a first ratio, where the first ratio is a ratio of the signal value related to the first image to the signal value related to the second image, the second image being generated by the imaging element when the second return light is captured when the opening amount of the shutter is the first opening amount.

2. The medical control device according to claim 1, wherein the second light is white light including a visible wavelength band.

3. The medical control device according to claim 1, further comprising:
a mode switching unit configured to switch an observation mode, wherein
the shutter control unit is configured to change the opening amount of the shutter to an opening amount according to the observation mode switched by the mode switching unit.

4. The medical control device according to claim 1, wherein
the imaging control unit further includes a gain control unit configured to adjust a gain of at least one of an analog gain and a digital gain for adjusting brightness of the first image,
the gain control unit is configured to use a gain larger than a gain reference value as the at least one gain when the brightness of the first image is darker than reference brightness, and
the shutter control unit is configured to set the opening amount of the shutter to an opening amount smaller than an opening amount reference value when the brightness of the first image is brighter than the reference brightness.

5. The medical control device according to claim 1, further comprising:
a light source control unit configured to control an operation of a light source device configured to emit the first light and the second light, wherein
the light source control unit is configured to intermittently turn on the first light emitted from the light source device for each field for forming the first image at a predetermined cycle obtained by dividing a period of the field.

6. The medical control device according to claim 1, wherein
the first return light is first fluorescence based on a first fluorescence agent, and
the shutter control unit is configured to set the opening amount of the shutter to the first opening amount when the first return light is captured and set the opening amount of the shutter to the second opening amount when second fluorescence based on a second fluorescence agent emitting fluorescence weaker than the first fluorescence is captured.

7. The medical control device according to claim 1, further comprising an operation receiving unit configured to receive a user operation for setting the opening amount of the shutter.

8. The medical control device according to claim 1, wherein the shutter control unit is configured to control an opening amount of an electronic shutter in the imaging element.

9. A medical control device comprising:
an imaging control unit configured to control an operation of an imaging element and causes the imaging element to capture return light from an observation target irradiated with narrow band light, wherein
the imaging control unit includes a shutter control unit configured to change a signal value related to an image by controlling an opening amount of a shutter for imaging, the image being generated by the imaging element when the return light is captured, and
the shutter control unit is configured to set the opening amount of the shutter to a second opening amount smaller than a first opening amount to desaturate the signal value when the signal value is saturated in a case where the opening amount of the shutter is set to the first opening amount.

10. A medical observation system comprising:
an imaging element configured to capture first return light from an observation target irradiated with first light that is narrow band light and second return light from the observation target irradiated with second light having a wavelength band different from a wavelength band of the narrow band light; and
an imaging control unit configured to control an operation of the imaging element, wherein
the imaging control unit includes a shutter control unit configured to change a signal value related to a first image by controlling an opening amount of a shutter for imaging, the first image being generated by the imaging element when the first return light is captured, and
the shutter control unit is configured to change the opening amount of the shutter from a first opening amount to a second opening amount smaller than the first opening amount to set a ratio of the signal value related to the first image to a signal value related to a second image to a second ratio smaller than a first ratio, where the first ratio is a ratio of the signal value related to the first image to the signal value related to the second image, the second image being generated by the imaging element when the second return light is captured when the opening amount of the shutter is the first opening amount.

11. A medical observation system comprising:
an imaging element configured to capture return light from an observation target irradiated with narrow band light; and
an imaging control unit configured to control an operation of the imaging element, wherein
the imaging control unit includes a shutter control unit configured to change a signal value related to an image by controlling an opening amount of a shutter for imaging, the image being generated by the imaging element when the return light is captured, and
the shutter control unit is configured to set the opening amount of the shutter to a second opening amount smaller than a first opening amount to desaturate the signal value when the signal value is saturated in a case where the opening amount of the shutter is set to the first opening amount.
